(19) United States
(12) Patent Application Publication
(10) Pub. No.: US 2004/0181149 A1
(43) Pub. Date: Sep. 16, 2004

(54) Device and method for intraoperative navigation

(76) Inventors: Ulrich Langlotz, Ittigen (CH); Lutz-Peter Nolte, Thun (CH)

Correspondence Address: JONES DAY, 51 Louisiana Aveue, N.W, WASHINGTON, DC, 20001-2113, US

(21) Appl. No.: 10/471,000

(22) PCT Filed: Feb. 7, 2001
(86) PCT No.: PCT/CH01/00087

(51) Int. Cl. A61B 6/12; A61B 17/88; A61F 2/46

(52) U.S. Cl. 600/431; 623/902; 606/102

(57) ABSTRACT

The invention relates to a device for intraoperative navigation and placing of a medical implant, in particular, a prosthesis, using a mobile medical imaging device A) a position determination device (15) for the spatial position measurement of the reference elements (1) relative to a spatially-fixed coordinate system (13); B) a mobile medical imaging device (3), comprising a radiation source (10) and a receiver unit (11), C) a computer (6) connected to the position determination device (15) and comprising a screen (7) whereby D) radiation source (10) and receiver unit (11) are connected to an imaging unit (18), which may be displaced about the room in a manner as to be fixed relative to each other and E) a reference element (1) is fixed to each of the imaging device (18), the bones (2) for treatment, and the surgical instrument (14).

The invention relates to a device for intraoperative navigation in surgery, particularly for placing a medical implant or prosthesis according to the introductory clause of patent claim 1 and to a method for navigation in surgery according to the introductory clause of patent claim 5.

Computers are often used at the present in surgery for image processing and position determination devices are employed for intraoperative position measurement of surgical instruments, tools and implants as well as the position of relevant bones or bone fragments of the patient. Such devices (CAS systems=computer-assisted surgery systems) serve, for one thing, to show the surgeon on a screen X-ray images taken before or during the operation in case of minimal invasive operations where the surgeon has no direct line of sight because of the small incisions in the tissue around the relevant bone. Should image data gathering be performed via computer whereby the images can be presently stored in digital form as a matrix of typically 128² to 1024² pixels, then pictorial representation of bones or bone fragments can be produced from these X-ray images on a screen or through other projection means, such as full views, perspective illustrations or sectional views.

Should the implantation of the prosthesis take place with the aid of computer-assisted navigation, then, the images must be registered in-situ before the operation with the patient's bone or the bone fragment to be treated whereby the images are used in the planning of the surgery. The registration process serves thereby to determine a geometric transformation between the position of points on the actual bone of the patient relative to the three-dimensional coordinate system in the operating room and the position of identical points on a virtual bone stored in the computer in form of a data set relative to the coordinate system of the images.

One method for implanting a knee prosthesis by means of computer-assisted navigation is disclosed in U.S. Pat. No. 5,682,886. Images of the pertinent body portions of the patient are generated by means of a radiation source and a receiver and then stored in the computer as a data set. A three-dimensional computer model of the recorded body section is generated by means of a computer. Employed as radiation source are preferably a CT scanner, an MRI device (magnetic resonance imaging), or an X-ray source. A conventional scanning protocol is employed in the use of the preferred CT scanner to gather image data. The images generated by computed tomography are two-dimensional, cross-sectional images of the bone or the body portion. Such cross-sectional images are taken through this protocol in a plurality of axially juxtaposed layers whereby the layer thickness is 1.5 mm, for example. The number of images to be taken depends on the length of the bone itself. The operator produces subsequently a three-dimensional computer model, preferably a surface reconstruction, which must be registered with the actual bone or body portion of the patient before the start of the surgery. This registration can be performed by scanning several anatomical landmarks on the body of the patient and by determining the corresponding points on the screen. After the completed registration, the measured position of the respectively used surgical instrument or tool is illustrated in a perspective view or as a section of the computer model of the bone so that the surgeon can optically observe on the screen the relative position, e.g. the invisible bones in-situ and instrument parts. A disadvantage in this known method is the costly and complicated method of producing the pictorial representation of the bone via computed tomography.

The invention has the aim to find a remedy in this regard. The invention is based on the object to provide a device and a method for surgical navigation that is based on a reference coordinate system formed by only a few anatomical landmarks. Expenditures for the determination of a reference system for surgical navigation are considerably reduced through the inventive method wherein the computer-assisted navigation of the surgical instrument used in operations can be performed with clearly lower costs.

The invention achieves the stated object with a device for intraoperative navigation having the characteristics of claim 1 and with a method for intraoperative navigation having the characteristics of claim 5.

The device according to the invention serves for intraoperative navigation in surgery, particularly placing of a medical implant or a prosthesis with the aid of a medical imaging device and it comprises essentially a mobile medical imaging device having at least one radiation source and at least one receiving unit for rays emitted by at least one radiation source, and at least one surgical instrument or implant, a position determination device and a computer connected to said position determination device, as well as a screen. At least one radiation source as well as at least one receiving unit are fixed relative to each other and are connected to a mobile receiving unit in the operating room. A reference element is attached to said receiving unit and to at least one surgical instrument, respectively, whereby said reference element's spatial position and orientation can be determined relative to a spatially-fixed coordinate system by means of a position determination device. The computer comprises furthermore a screen whereon there can be displayed by means of the imaging device preoperative or intraoperative images taken or full views, perspective illustrations, or sectional views of virtual bones or bone fragments stored in the processor or memory as data set. The surgeon obtains with the aid of surgical navigation numeric and/or graphic feedback about angles and positions or depths of the surgical instrument and possible superposition of the instrument position with a medical image data set. This medical image data set can be a representation of a bone or a bone fragment and it can consist, for example, of intraoperative X-ray images taken, and it can be stored in the memory of the computer in form of a data set.

In the preferred embodiment of the inventive device, the mobile imaging device comprises a wheeled frame movable at floor level of an operating room and an imaging unit which is movable relative to the spatially-fixed coordinate system in three superposed axial directions and which is rotatable about said axes.

The intraoperative navigation together with the employment of surgical instruments and feedback about the position of the surgical instrument relative to the bone require a reference system connected closely with the bone whereby the position of said reference system must be defined in the spatially-fixed coordinate system. Said surgical instruments can be pinpointed relative to their position in the spatially-fixed coordinate system by the position determination device. This referencing of the spatially-fixed coordinate system, together with the reference system on the bone, may be conducted at low costs through the method according to the invention.

The inventive method for navigation in surgery, particularly placing of a medical implant or prosthesis, includes essentially the following steps:

A) Defining and measuring of three reference points arranged non-linear on a bone of a patient. The position of these reference points may be determined percutaneously by means of a pointer. In place of a pointer, there can also be employed an ultrasonic device or some other device for three-dimensional locating of points, such as an X-ray device, for example. A reference element is fastened to this device (pointer, ultrasonic device or X-ray device) to measure the reference points relative to a spatially-fixed coordinate system whereby the position of said reference element can be detected relative to the spatially-fixed coordinate system by the position determination device and the computer. The position of the reference point relative to the spatially-fixed coordinate system can be determined from the known position of the pointer tip or the ultrasonic source or the plane of the image in the image-producing X-ray process relative to the respective reference system.

B) Creating a reference system from the measured reference points according to step A). The reference points are anatomical points so that the anatomy of the bone is known relative to the reference system.

C) Performing a surgical operation step with a surgical instrument, implant or prosthesis.

D) Measuring of the position of the surgical instrument, the implant, or the prosthesis relative to its position to the spatially-fixed coordinate system and transferring the position into the reference system.

In the preferred embodiment of the inventive method there is one axis X′, Y′ of the reference system identical to the longitudinal axis, and the other axis X′, Y′ is identical to the transverse axis of the patient whereby the sagittal plane, the transverse plane, and the coronal plane can be determined.

The advantages achieved by the invention are essentially shown in the fact that:

the radiation exposure is considerably reduced and the cost are considerably reduced thereby as well; and

the reference system can be determined also without additional preoperative steps (e.g. establishing a preoperative image data set or planning).

The invention and the development of the invention are described now in the following with the aid of the partially schematic illustrations of several embodiment examples.

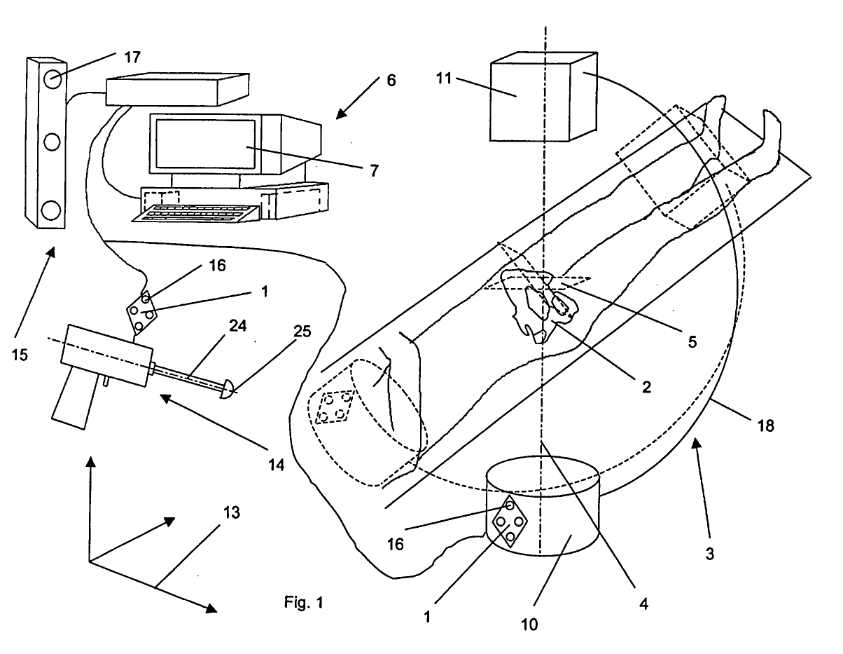

FIG. 1 shows one embodiment of the inventive device for intraoperative navigation in surgery;

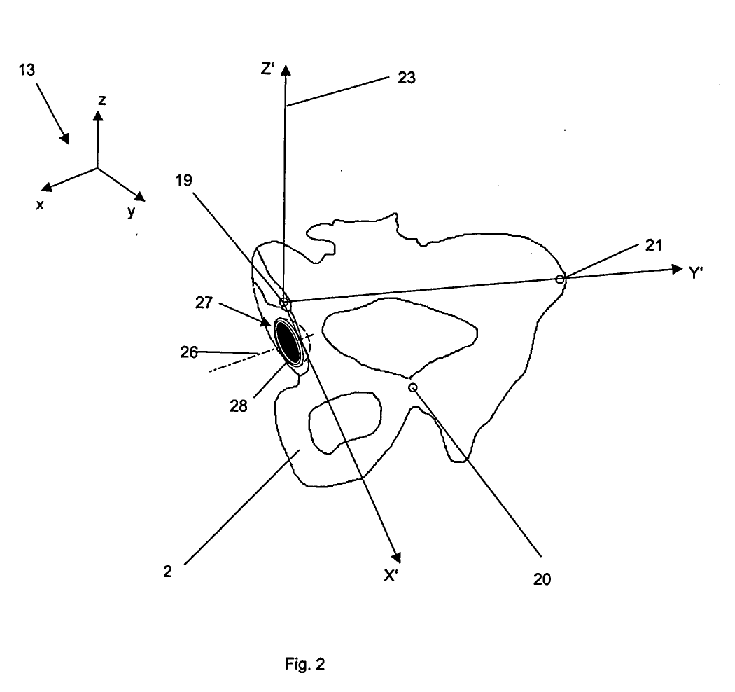

FIG. 2 shows a hipbone with the reference system determined according to the inventive method;

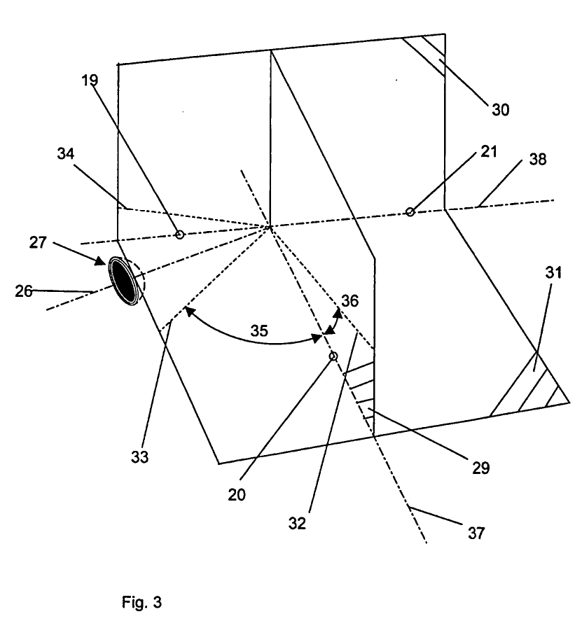

FIG. 3 shows the definition of the angles of inclination and anteversion; and

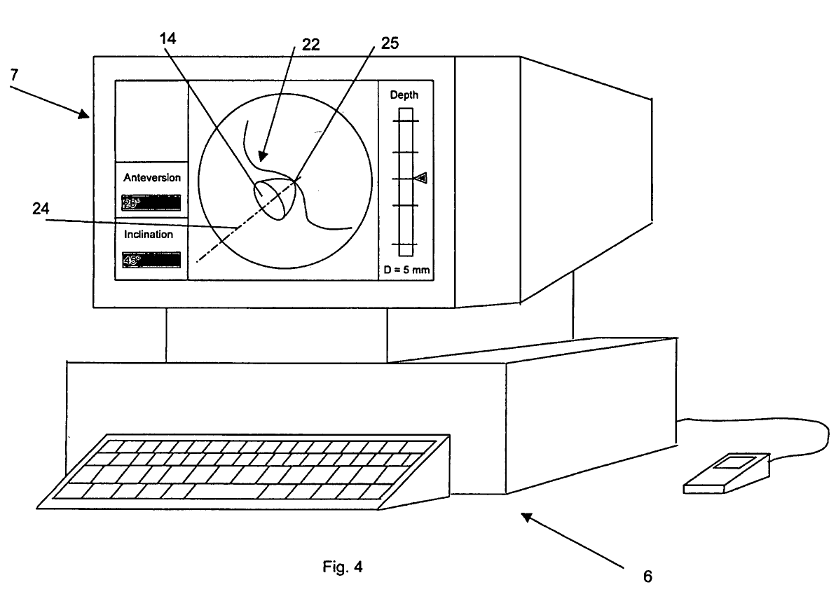

FIG. 4 shows the display of the axis of a surgical instrument in the instance of an acetabulum operation with surgical navigation.

FIG. 1 shows a device for surgical navigation in the example of an implant of an artificial hip socket with the aid of a mobile medical imaging device 3. Such an imaging device 3, for instance an X-ray device, comprises essentially one or several radiation sources 10 and one or several receivers 11, which are arranged along a central axis 4 and which have a projection plane 5. The device comprises essentially a position determination device 15 for the spatial measurement of reference elements 1 relative to a spatially-fixed three-dimensional coordinate system 13, a computer 6, which includes display means 7 and which is connected to said position determination device 15, and it comprises reference elements 1 measurable by the position determination device 15. Such reference elements 1 are attached to the imaging device 3 and to the corresponding surgical instrument 14. The reference elements 1 comprise four markers 16 recorded by the cameras 17 of the position determination device 15 so that there can be determined the position and the spatial orientation of the reference elements 1 relative to the coordinate system 13 in-situ. The position of the acetabulum 27, the direction of axis 24 of the surgical instrument 14, and the position of its tip 25 can be determined relative to the coordinate system 13 through measuring of the position and the spatial orientation of the reference elements 1, and computed and shown on the display means 7 can be from this the numeric values of the relevant momentarily in-situ set angles of anteversion 36 (FIG. 3) and inclination 35 (FIG. 3) of axis 24 of the surgical instrument 14. During the operation, the surgeon can make the correction of the direction of axis 24 of the surgical instrument 14 based on the size of the angles of anteversion 36 and inclination 35 shown on the display means 7 or their deviation to a possible plan. An evacuation tool to work on the acetabulum is exemplary shown here as a surgical instrument 14.

The reference elements 1 include at least three markers 16 that are not arranged in a straight line. The markers 16 as well as the position-finding means 17 of the position determination device 17 may be in the form of acoustic or electro-magnetic means in their effect whereby the embodiment shown here has an opto-electric position determination device 15.

FIG. 2 shows a hipbone 2 with the acetabulum 27 and an artificial joint socket 28 with the axis 26 of the acetabulum 27 which extends through the center of the joint socket and is oriented perpendicular to the face of the joint socket. According to the inventive method, the position of the three reference points 19, 20, 21 on the hipbone 2 is measured relative to a coordinate system 13. Suitable as reference points 19, 20, 21 on the hipbone are, for example:

reference point 19: right spina iliac anterior superior;

reference point 20: center of pubis; and

reference point 21: left spina iliac anterior superior.

The reference system can then be established as coordinate system 23 from the coordinates of the three reference points 19, 20, 21 whereby its x-axis X′ corresponds to the longitudinal axis 37 of the patient (FIG. 3) and whereby the patient's y-axis Y′ corresponds to the transverse axis 38 of the patient. The relevant angles of inclination and anteversion can be determined by means of said coordinate system 23.

The position of the three reference points 19, 20, 21 can be percutaneously determined by means of a pointer (not illustrated) whose tip is measured spatially. An ultrasonic device or an image-producing device, e.g. an X-ray device, may be employed in place of said pointer.

FIG. 3 serves to explain the two angles of anteversion 36 and inclination 35 within a reference system, which includes the sagittal plane 29, the transverse plane 30 and the coronal plane 31, whereby the longitudinal axis 37 of the patient lies in the coronal plane 31.

Axis 26 of the acetabulum 27 is projected by a first projection line 32 into the sagittal plane 29, through a second projection line 33 into the coronal plane 31, and through a third projection line 34 into the transverse plane 30. The operative definition is illustrated here in regard to the definition of anteversion 36 and inclination 35. According to D. W. Murray “The Definition and Measurement of Acetabular Orientation” in The Journal of Bone and Joint Surgery, 1993, page 228 and following pages there are three different definitions common for anteversion and inclination:

a) Operative Definition:

The operative inclination 35 is the angle between the second projection line 33 and the sagittal plane 29, while the operative anteversion 36 is the angle between the first projection line 32 and the longitudinal axis 37 of the patient.

b) Anatomical Definition:

The anatomical inclination is the angle between the axis 26 of the acetabulum and the longitudinal axis 37 of the patient, while the anatomical anteversion is the angle between the third projection line 34 and the transverse axis 38.

c) Radiographical Definition:

The radiagraphical (X-ray) inclination is the angle between the second projection line 33 and the longitudinal axis 37 of the patient, while the radiagraphical anteversion 36 is the angle between the axis 26 of the acetabulum 27 and the coronal plane 31.

These differently defined angles can also be fittingly converted in a corresponding manner according to D. W. Murray “The Definition and Measurement of Acetabular Orientation” in The Journal of Bone and Joint Surgery, 1993, page 228 and following pages.

FIG. 4 shows an embodiment of means suitable for intraoperative surgical navigation in the intraoperative use of the angle display of anteversion 36 (FIG. 3) and inclination 35 (FIG. 3) on the basis of the reference system determined by means of the inventive method. These means comprise essentially a computer 6 and display means 7 connected thereto. The display means 7 consist here of a screen, but they can include other embodiments, for instance a head-mounted display. A graphic illustration of the surgical instrument 14 is shown on the display means 7 having an axis 24 and a tip 25. Furthermore, the numerical values of the relevant angles of inclination 35 and anteversion 36 are shown on the display means 7. In addition, a scale can be inserted in said display means 7 to display the depth between the surface of the acetabulum and the tip 25 of the surgical instrument 14. Should an image-producing device be intraoperatively employed, e.g. a mobile X-ray device 3 (FIG. 1), then a projection of the acetabulum 22 can be additionally shown on the display means 7.

1. A device for intraoperative navigation in surgery, particularly placing of a medical implant or prosthesis comprising
A) a mobile medical imaging device (3) having at least one receiving unit (11) for rays emitted by the radiation source (10);
B) at least one surgical instrument (14), implant or prosthesis onto which a reference element is fastened;
C) a position determination device (15) for spatial position measurement of the reference elements (1) relative to a spatially-fixed coordinate system (13); and
E) a computer (6) which is connected to said position determination device (15) and which includes display means (7),
characterized in that
F) at least one radiation source (10) and at least one receiving unit (11) are fixed relative to each other and to an imaging unit (18), which may be displaced about the room, and whereby an additional reference element (1) is fastened to said imaging device (18):

2. A device according to claim 1, whereby the mobile imaging device (3) comprises a wheeled frame movable at the floor level of an operating room.

3. A device according to claim 1 or 2, whereby the imaging unit (18) can be moved in three perpendicular superposed axial directions relative to the spatially-fixed coordinate system (13).

4. A device according to one of the claims 1 through 3, whereby said imaging unit (18) is rotatable about three perpendicular superposed axes relative to the spatially-fixed coordinate system (13).

5. A method for navigation in surgery, particularly placing of a medical implant or prosthesis with the following steps:
A) defining of three reference points (19, 20, 21) arranged not in a straight line on a bone (2) of a patient;
B) measuring said reference points (19, 20, 21) relative to their position within a coordinate system (13);
C) creating a reference system (23) from the reference points (19, 20, 21) measured in step B); and
D) conducting a step in an operation with a surgical instrument (14);
E) measuring the position of the surgical instrument (14) relative to its position in relation to said coordinate system (13),
characterized in that
F) the reference points (19, 20, 21) are anatomically marked points so that the anatomy of the bone (2) is known relative to the reference system (23).

6. A method according to claim 5, wherein one axis (X′, Y′) of the reference system (23) is identical to the longitudinal axis (37) and the other axis (X′, Y′) is identical to the transverse axis (38) of the patient by which the sagittal plane (29), the transverse plane (30) and the coronal plane (31) can be determined.

7. A method according to claim 6, wherein the position of the axis (24) of a surgical instrument (14), an implant or prosthesis can be determined within said reference system (23) by two angles which extend between the axis (24) and the planes of the reference system (23) relative to the coordinate axes.

8. A method according to claim 7, wherein the angles (α,β) represent the operative inclination (35) and the operative anteversion (36) whereby said operative inclination (35) is the angle between the axis (24) and the sagittal plane, (29) and the operative anteversion (36) is the angle between the straight line of the projection (33) of the axis (24) on the sagittal plane (29) and the longitudinal axis (37) of a patient.
